(19) United States
(12) Patent Application Publication
(10) Pub. No.: US 2019/0362531 A1
(43) Pub. Date: Nov. 28, 2019

(54) SYSTEMS AND METHODS FOR GENERATING AVATARS USING GENOTYPING DATA

(71) Applicant: Orig3n, Inc., Boston, MA (US)

(72) Inventors: Robin Y. Smith, Boston, MA (US); Marcie A. Glicksman, Boston, MA (US)

(21) Appl. No.: 16/484,162

(22) PCT Filed: Feb. 14, 2018
(86) PCT No.: PCT/US2018/018103

§ 371 Date: Aug. 7, 2019

Related U.S. Application Data
(60) Provisional application No. 62/458,949, filed on Feb. 14, 2017.

(51) Int. Cl. G06T 13/40; G16B 20/20; G16B 5/00; G16B 45/00; C12Q 1/6827; A63F 13/69; A63F 13/79; A63F 13/58; A63F 13/825

(52) U.S. Cl. CPC G06T 13/40; G16B 20/20; G16B 5/00; G16B 45/00; A63F 13/825; A63F 13/69; A63F 13/79; A63F 13/58; C12Q 1/6827

(57) ABSTRACT

Presented herein are methods and systems for using genotyping data of an individual to create and/or modify in-game avatars. Genotyping data is used to create and update avatar objects. Avatar objects are data structures that comprise data, based on genotyping data, that define individual avatars and can be exported for import into a game to generate an avatar. Traits of an individual are characterized by feature values included in an avatar object. Avatars generated from avatar objects may appear to physically resemble an individual or may have an appearance that fits the thematic elements of a game while still reflecting the phenotype of the individual (based on the individual's genotyping data). Avatars may be used in games for entertainment, for interactive or realistic health tracking, simulations, simulated work environments, or other similar purposes.

### CROSS REFERENCE TO RELATED APPLICATIONS

This application claims priority to and the benefit of U.S. Provisional Patent Application No. 62/458,949 filed on Feb. 14, 2017, the contents of which are hereby incorporated by reference in their entirety.

### FIELD OF INVENTION

This invention generally relates to systems and methods for generating and modifying avatars that reflect traits of individuals based on genotyping data.

### BACKGROUND

Electronic gaming systems and games frequently allow different players to select avatars in order to add some level of customizability to a game and additionally to allow different players to distinguish between each other in a multiplayer game or game mode. Selectable characters or avatars may be based on characters within a game or a game franchise. Custom design engines in games allow players to customize a range of features for a character that are then imported into the games such that players play as their customized character. More recently, players have been able to use custom design engines to design characters with realistic or semi-realistic features such that players may design characters that resemble themselves.

Custom design engines with realistic or semi-realistic feature choices allow players to choose features from a range of predetermined options in order to produce characters that resemble the player. Features such as hair length and color, eye color, gender, height, weight, facial bone structure, and skin tone are commonly selectable. In certain games, players may select intangible traits for their characters when designing them. Intangible traits may include intelligence, endurance, strength, speed, and dexterity. Using such custom design engines, players can have complete control when designing their characters. Frequently, this leads an individual to design a character that reflects their personal attributes (tangible and intangible) by self-assessing which selectable features most resemble the player.

Creating an avatar that resembles an individual by selecting a large number of features for the avatar can be time consuming and yield an unsatisfactory result. For example, due to inaccurate self-assessment by the player, the avatar may not have attributes that accurately reflect reality. A high level of realism is a critical aspect in many games and especially games that are used to track real world developments (e.g., personal wellness based games). There is a need for systems and methods of creating avatars, for use in games, that accurately reflect the biological characteristics and traits of an individual. Additionally, there is a need for systems and methods that facilitate automatic generation of avatars that accurately reflect the biological characteristics and traits of an individual.

### SUMMARY

Presented herein are methods and systems for using genotyping data of an individual to create and/or modify in-game avatars. Genotyping data is used to create and update avatar objects. Avatar objects are data structures that comprise data, based on genotyping data, that define individual avatars. An individual's traits are characterized by feature values included in an avatar object. Avatar objects can be exported for import into a game to generate an avatar. Avatars generated from avatar objects may appear to physically resemble an individual or may have an appearance that fits the thematic elements of a game while still reflecting the phenotype of the individual (based on the individual's genotyping data). Avatars may be used in games for entertainment, for interactive or realistic health tracking, simulations, simulated work environments, or other similar purposes. In certain embodiments, avatars that reflect an individual's biological traits can be automatically generated from avatar objects.

Genotyping data provides a quantitative and/or semi-quantitative assessment of an individual's phenotype (i.e., biological traits). Until recently, characterizing a genome was prohibitively expensive such that very few individual genomes had been fully or partially characterized. Development of cost-effective equipment and procedures for genotyping have made personal genotyping feasible. Thus, genotyping data for large numbers of individuals can be easily obtained from biological samples provided by the individuals. Genotyping data includes measurements of single nucleotide polymorphisms (SNPs) whose particular variant in an individual determines, at least partially, a biological characteristic or trait of the individual. Variants of SNPs may allow a quantitative determination (such as an individual being lactose intolerant or not) or a semi-quantitative determination (in that an individual is more or less likely to have a particular characteristic or trait) to be made.

Genotyping data may be stored in a personal genetic profile assessment of an individual. Personal genetic profile assessments are data structures with a hierarchy that organizes genes, SNPs, and SNP variants based on their relationship to particular biological characteristics. Examples include (i) nutritional characteristics (e.g., the way in which an individual's body processes different foods and nutrients), (ii) skin health, (iii) physical fitness, and (iv) personal behavior tendencies (e.g., empathy, risk of addiction, and tolerance for stress and pain). In certain embodiments, an avatar object, a data structure used to generate an avatar of an individual that reflects the biological traits of the individual, is created using the data stored in a personal genetic profile assessment.

Avatars that reflect an individual's biological characteristics and traits can be generated (e.g., automatically) from avatar objects that store data derived from genotyping data for the individual. Avatar objects comprise avatar features, which comprise feature values. Avatar features correspond to particular biological traits that individuals possess. Feature values are values that characterize a particular trait corresponding to an avatar feature for individuals. Feature values may be used to characterize a physical trait, such as hair color or height. Feature values may be used to characterize an intangible trait, such as dexterity or metabolism. A set of avatar features, each associated with a feature value, collectively defines the particular biological traits that are reflected in an individual's avatar, as determined based on the individual's genotyping data. Each feature value can be determined from a portion of an individual's genotyping data, such as a measurement of a variant (e.g., a specific variation of a specific SNP). Feature avatars and/or feature values may be standardized (e.g., stored in a standardized hierarchy with standardized values) such that avatar objects containing standardized data can be exported or ported to a third party for generation of individual avatars. In certain embodiments, avatars generated from avatar objects are suitable for graphical rendering (e.g., for view by the individual corresponding to the avatar). In certain embodiments, avatars are suitable for modeling or simulations (e.g., behavior modeling) with or without graphical rendering.

In certain embodiments, developers use a back end system and/or method to create avatar features. Generic avatar features created using such a back end may have a plurality of feature values associated with them, of which each is also associated with a variant object, associated with a generic SNP object, corresponding to a variant of a SNP that an individual may possess. Thus, in some embodiments, each variant that an individual is determined to have (e.g., by genotyping) will have a corresponding feature value that is assigned to the relevant avatar feature in an individual's avatar object. In this way, an individual's personal genetic profile assessment can be used to automatically populate an individual's avatar object such that the avatar object accurately reflects the individual's biological traits.

In one aspect, the invention is directed to a method for creating (and/or updating) an avatar object based on genotyping data, the method comprising: receiving (and/or accessing), by a processor of a computing device, genotyping data (e.g., a personal genetic profile assessment) generated using a biological sample of one or more individuals (e.g., one or more players); identifying, by the processor, one or more portions of the genotyping data that correspond to a distinct biological characteristic or trait (e.g., a variant of a SNP); determining, by the processor, an avatar feature and a feature value of the avatar feature based on the one or more portions of the genotyping data; creating (and/or updating), by the processor, an avatar object for each of the one or more individuals, wherein the avatar object comprises each determined avatar feature and the feature value of the avatar feature for each of the one or more individuals; and storing, by the processor, the avatar object for further use by a person (e.g., in representing the one or more individuals) (e.g., in playing a game, running a simulation (e.g., a simulated work environment), in online matchmaking, in digital health tracking (e.g., interactive health tracking), or running other software).

In certain embodiments, the method comprises identifying, by the processor, at least one avatar feature that corresponds to at least one of the one or more portions of the genotyping data. In certain embodiments, the genotyping data has been generated from one or more genotyping measurements of one or more SNPs.

In certain embodiments, the receiving (and/or accessing) step comprises accessing or extracting (e.g., automatically, by the processor) genotyping data from a personal genetic profile assessment. In certain embodiments, the determining step comprises determining, by the processor, the feature value by selecting a numerical value from a predefined range of values (e.g., a numerical value on a scale, e.g., a scale from 1-20 or 1-10 or 1-100). In certain embodiments, the determining step comprises: determining, by the processor, the feature value based on a plurality of the one or more portions of the genotyping data. In certain embodiments, the method comprises determining, by the processor, a sub-range from the one or portions of genotyping data, wherein the feature value is selected from the sub-range. In certain embodiments, the determining step comprises automatically determining, by the processor, the feature value based on a particular variant of a SNP that the individual has.

In certain embodiments, the avatar object is for use in a member selected from the group consisting of playing a game, running a simulation (e.g., a simulated work environment), in online matchmaking, in digital health tracking (e.g., interactive health tracking), and a virtual reality experience. In certain embodiments, the avatar object obfuscates identities of the one or more individuals (e.g., to protect privacy of the one or more individuals).

In another aspect, the invention is directed to a method for linking feature values of avatar features to personal genetic profile products, the method comprising: presenting, by a processor of a computing device, a graphical user interface element (e.g., widget) for creation of an avatar feature that defines a portion of an avatar for use by an individual in a game; receiving, by the processor, via the graphical user interface element, the avatar feature; receiving, by the processor, via the graphical user interface element, a developer selection of one or more stored objects (e.g., gene objects, SNP objects, variant objects) that correspond to one or more genomic constituents (e.g., genes, SNPs, variants), wherein the one or more genomic constituents correspond to a biological trait corresponding to the avatar feature; creating and/or updating, by the processor, the avatar feature; associating, by the processor, the avatar feature with the one or more stored objects; and storing, by the processor, the avatar feature and the association(s) between the avatar feature and the one or more stored objects for further retrieval and/or updating (e.g., in generating an avatar object and displaying an in-game avatar for an individual).

In certain embodiments, the avatar feature comprises one or more feature values associated with the avatar feature, wherein the one or more feature values are used to graphically render an avatar from an avatar object comprising the avatar feature or to provide a quantitative value of an individual ability for use in game play.

In certain embodiments, the method comprises the step of: associating, by the processor, each of the one or more feature values with a distinct variant object, wherein each distinct variant object is associated with a common SNP object. In certain embodiments, the one or more stored objects are one or more SNP objects and the one or more genomic constituents are one or more SNPs.

In certain embodiments, the association(s) between the avatar feature and the one or more stored genomic objects are indirect association(s).

In certain embodiments, the method comprises: receiving, by the processor, a developer selection of one or more avatar feature collection objects, wherein the one or more avatar feature collection objects correspond to a set of related biological characteristics; and associating, by the processor, the avatar feature with the one or more avatar feature collection objects.

In another aspect, the invention is directed to a system for creating (and/or updating) an avatar object based on genotyping data, the system comprising: a processor; and a non-transitory computer readable medium having instructions stored thereon, wherein the instructions, when executed by the processor, cause the processor to: receive (and/or access) genotyping data (e.g., a personal genetic profile assessment) generated using a biological sample of one or more individuals (e.g., one or more players); identify one or more portions of the genotyping data that correspond to a distinct biological characteristic or trait (e.g., a variant of a SNP); determine an avatar feature and a feature value of the avatar feature based on the one or more portions of the genotyping data; create (and/or update) an avatar object for each of the one or more individuals, wherein the avatar object comprises each determined avatar feature and the feature value of the avatar feature for each of the one or more individuals; and store the avatar object for further use by a person (e.g., in representing the one or more individuals) (e.g., in playing a game, running a simulation (e.g., a simulated work environment), in online matchmaking, in digital health tracking (e.g., interactive health tracking), or running other software).

In certain embodiments, the instructions, when executed by the processor, cause the processor to: identify at least one avatar feature that corresponds to at least one of the one or more portions of the genotyping data. In certain embodiments, the genotyping data has been generated from one or more genotyping measurements of one or more SNPs.

In certain embodiments, the instructions, when executed by the processor, cause the processor to receive (and/or access) the genotyping data by accessing or extracting the genotyping data from a personal genetic profile assessment. In certain embodiments, the instructions, when executed by the processor, cause the processor to determine the feature value by selecting a numerical value from a predefined range of values (e.g., a numerical value on a scale, e.g., a scale from 1-20 or 1-10 or 1-100). In certain embodiments, the instructions, when executed by the processor, cause the processor to determine the feature value based on a plurality of the one or more portions of the genotyping data. In certain embodiments, the instructions, when executed by the processor, cause the processor to determine a sub-range from the one or portions of genotyping data, wherein the feature value is selected from the sub-range. In certain embodiments, the instructions, when executed by the processor, cause the processor to determine the feature value (e.g., automatically) based on a particular variant of a SNP that the individual has.

In certain embodiments, the avatar object is for use in a member selected from the group consisting of playing a game, running a simulation (e.g., a simulated work environment), in online matchmaking, in digital health tracking (e.g., interactive health tracking), and a virtual reality experience. In certain embodiments, the avatar object obfuscates identities of the one or more individuals (e.g., to protect privacy of the one or more individuals).

In another aspect, the invention is directed to a system for linking avatar features to stored genomic objects (e.g., gene objects, SNP objects, variant objects), the system comprising: a processor; and a non-transitory computer readable medium having instructions stored thereon, wherein the instructions, when executed by the processor, cause the processor to: present a graphical user interface element (e.g., widget) for creation of an avatar feature that defines a portion of an avatar for use by an individual in a game; receive, via the graphical user interface element, the avatar feature; receive, via the graphical user interface element, a developer selection of one or more stored objects (e.g., gene objects, SNP objects, variant objects) that correspond to one or more genomic constituents (e.g., genes, SNPs, variants), wherein the one or more genomic constituents correspond to a biological trait corresponding to the avatar feature; create and/or update the avatar feature; associate the avatar feature with the one or more stored objects; and store the avatar feature and the association(s) between the avatar feature and the one or more stored objects for further retrieval and/or updating (e.g., in generating an avatar object and displaying an in-game avatar for an individual).

In certain embodiments, the avatar feature comprises one or more feature values associated with the avatar feature, wherein the one or more feature values are used to graphically render an avatar from an avatar object comprising the avatar feature or to provide a quantitative value of an individual ability for use in game play.

In certain embodiments, the instructions, when executed by the processor, cause the processor to: associate each of the one or more feature values with a distinct variant object, wherein each distinct variant object is associated with a common SNP object. In certain embodiments, the one or more stored objects are one or more SNP objects and the one or more genomic constituents are one or more SNPs. In certain embodiments, the association(s) between the avatar feature and the one or more stored genomic objects are indirect association(s). In certain embodiments, the instructions, when executed by the processor, cause the processor to: receive a developer selection of one or more avatar feature collection objects, wherein the one or more avatar feature collection objects correspond to a set of related biological characteristics; and associate the avatar feature with the one or more avatar feature collection objects.

### Definitions

In order for the present disclosure to be more readily understood, certain terms used herein are defined below. Additional definitions for the following terms and other terms may be set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Genotyping data: As used herein, the term “genotyping data” refers to data obtained from measurements of a genotype. Measurements of a genotype performed on a biological sample identify the particular nucleotide(s) (also referred to as “bases”) that is/are incorporated at one or more particular positions in genetic material extracted from the biological sample. Accordingly, genotyping measurements for a particular individual are measurements performed on a biological sample of from the individual, and which identify the particular nucleotides present at one or more specific positions within their genome.

Genotyping data may be measurements of particular genes (e.g., portions of an individual's genetic sequence, e.g., DNA sequence), or SNPs. For example, a genotyping measurement of a particular SNP for an individual identifies the particular variant of that SNP that the individual has. A genotyping measurement of a particular gene for an individual identifies the particular nucleotides that are present at one or more locations within and/or in proximity to the gene for the individual. For example, genotyping measurements of a particular gene may identify the particular variants of one or more SNPs associated with a particular gene.

In certain embodiments, genotyping data is obtained from a multi-gene panel. In certain embodiments, genotyping data is obtained from assays (e.g., TaqMan™ assays) that detect one or more specific variants of specific SNPs. In certain embodiments, genotyping data is obtained from genetic sequencing measurements. In certain embodiments, genotyping data is generated in response to a purchase or request by an individual. In certain embodiments, genotyping data comprises data for a portion of a genotype (e.g., of an individual). In certain embodiments, genotyping data comprises all available measurements of a genotype (e.g., of an individual).

Individual: As used herein, the term “individual” refers to a person who uses an avatar. In certain embodiments, the avatar is used in games in which a customizable or personalizable character can be designed and/or created. In certain embodiments, an individual uses his or her avatar in a digital environment (e.g., in a simulation, in virtual reality, in a game, for online matchmaking, for interactive or realistic health tracking, or in simulated work environments). As used herein, the terms “user” and “individual” are used interchangeably.

Developer: As used herein, the term “developer” refers to a person, company, or organization that creates avatar objects or makes, sells, or operates software that generates avatars from avatar objects. In certain embodiments, a developer also genotypes a biological sample in response to an assessment corresponding to a product being purchased or made accessible to an individual.

Variant: As used herein, the term “variant” refers to a specific variation of a specific SNP occurring in the genetic material of a population. In certain embodiments, a variant is a specific combination of a first allele of a first copy of an individual's genetic material (e.g., corresponding to an individual's paternal DNA) and a second allele of a second copy of an individual's genetic material (e.g., corresponding to an individual's maternal DNA), as occurs in diploid organisms (e.g., humans).

Qualifier: As used herein, the term “qualifier” refers to a classification (e.g., a label) of a particular variant of a given SNP. The qualifier associated with a given variant is the particular classification (e.g., label) of that variant. For example, a given variant may be associated with a particular qualifier of a predefined set of possible qualifiers. For example, a given variant may be associated with a qualifier selected from a group of labels such as “Adapt,” “Normal,” and “Gifted.” In certain embodiments, for a given variant of a given SNP, a qualifier corresponds to a classification of the given variant based on (i) the prevalence of the given variant within a population (e.g., if the variant is common, e.g., if the variant is rare) and/or (ii) a trait associated with the variant. For example, a common variant may be associated with the qualifier “Normal”. A rare variant that confers a disadvantageous trait, such as a predisposition to high cholesterol, may be associated with the qualifier “Adapt” (e.g., classified as rare and disadvantageous). A rare variant that confers an advantageous trait, such as a predisposition to lower cholesterol, may be associated with the qualifier “Gifted” (e.g., accordingly, the variant is classified as rare and advantageous).

Variant object: As used herein, the term “variant object” refers to a data structure corresponding to (e.g., that is used to represent) a specific variant of a physical gene within a given genome (e.g., the genome of a human).

SNP object: As used herein, the term “SNP object” refers to a data structure corresponding to (e.g., that is used to represent) a specific single nucleotide polymorphism (SNP). In certain embodiments, a SNP object comprises a SNP reference that identifies the specific SNP to which the SNP object corresponds. The SNP reference may be an alphanumeric code such as an accepted name of the SNP or other identifying mark or label capable of being stored electronically. The SNP reference may be an alphanumeric code such as a National Center for Biotechnology Information (NCBI) database reference number.

Gene object: As used herein, the term “gene object” refers to a data structure corresponding to (e.g., that is used to represent) a specific physical gene within a given genome (e.g., the human genome).

Category: As used herein, the term “category” refers to a data structure corresponding to (e.g., that is used to represent) a particular health-related trait or characteristic.

Product, Genetic Profile Product, Personal Genetic Profile Product: As used herein, the terms “product,” “genetic profile product,” and “personal genetic profile product” refer to a data structure corresponding to (e.g., that is used to represent) a general class of health-related traits and/or characteristics. In certain embodiments, a product is associated with one or more categories that correspond to health-related traits and characteristics related to the general class of health-related traits and characteristics to which the product corresponds.

Avatar object: As used herein, the term “avatar object” refers to a data structure that comprises sub-structures that define an individual's avatar. In certain embodiments, an avatar object comprises avatar features that define different aspects of an avatar. Each avatar feature may have an associated feature value that is used in graphically rendering an avatar or providing a quantitative value of an individual's ability.

Personal Genetic Profile Assessment: As used herein, the term “personal genetic profile assessment” refers to a data structure (e.g., a hierarchy of data structures) corresponding to (e.g., that is used to represent) genomic information of a user for one or more general classes of health-related traits and/or characteristics. In certain embodiments, a personal genetic profile assessment of a user is generated by associating genotyping data of the user with premade (i.e., stored) generic personal genetic profile products. In certain embodiments, a user's personal genetic profile assessment is viewed using an assessment graphical user interface (“assessment GUI”) on a computing device (e.g., a smartphone).

Graphical Control Element: As used herein, the term “graphical control element” refers to an element of a graphical user interface element that may be used to provide user and/or individual input. A graphical control element may be a textbox, dropdown list, radio button, data field, checkbox, button (e.g., selectable icon), list box, or slider.

Associate, Associated with: As used herein, the terms “associate,” and “associated with,” as in a first data structure is associated with a second data structure, refer to a computer representation of an association between two or more data structures or data elements that is stored electronically (e.g., in computer memory).

Provide: As used herein, the term “provide”, as in “providing data”, refers to a process for passing data in between different software applications, modules, systems, and/or databases. In certain embodiments, providing data comprises the execution of instructions by a process to transfer data in between software applications, or in between different modules of the same software application. In certain embodiments, a software application may provide data to another application in the form of a file. In certain embodiments, an application may provide data to another application on the same processor. In certain embodiments, standard protocols may be used to provide data to applications on different resources. In certain embodiments, a module in a software application may provide data to another module by passing arguments to that module.

### BRIEF DESCRIPTION OF THE DRAWINGS

Drawings are presented herein for illustration purposes, not for limitation. The foregoing and other objects, aspects, features, and advantages of the invention will become more apparent and may be better understood by referring to the following description taken in conjunction with the accompanying drawings, in which:

FIG. 1 is a block diagram showing an organizational hierarchy of a personal genetic profile product, according to an illustrative embodiment of the invention;

FIG. 2 is a block diagram illustrating associations between different data structures in a personal genetic profile product, according to an illustrative embodiment of the invention;

FIG. 3 is a block diagram showing a process for creating a personal genetic profile assessment, according to an illustrative embodiment of the invention;

FIG. 4 is a portion of a text file comprising genotyping data, according to an illustrative embodiment of the invention;

FIG. 5 is a block diagram illustrating associations between different data structures in an avatar object for an individual, according to an illustrative embodiment of the invention;

FIG. 6 is a block diagram of a method for creating an avatar object for an individual based on genotyping data, according to an illustrative embodiment of the invention;

FIG. 7 is a block diagram illustrating associations between data structures in an individual's personal genetic profile assessment and data structures in an individual's avatar object, according to an illustrative embodiment of the invention;

FIG. 8 is a block diagram of a method for linking feature values of avatar features to personal genetic profile products (e.g., stored genomic objects comprised therein (e.g., variant objects)), according to an illustrative embodiment of the invention;

FIG. 9 is a block diagram illustrating associations between the feature values of a generic avatar feature and the variant objects of a generic SNP object, according to an illustrative embodiment of the invention;

FIG. 10 is a block diagram of an example network environment for use in the methods and systems described herein, according to an illustrative embodiment of the invention; and

FIG. 11 is a block diagram of an example computing device and an example mobile computing device, for use in illustrative embodiments of the invention.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION

It is contemplated that systems, devices, methods, and processes of the claimed invention encompass variations and adaptations developed using information from the embodiments described herein. Adaptation and/or modification of the systems, devices, methods, and processes described herein may be performed by those of ordinary skill in the relevant art.

Throughout the description, where articles, devices, and systems are described as having, including, or comprising specific components, or where processes and methods are described as having, including, or comprising specific steps, it is contemplated that, additionally, there are articles, devices, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim. Headers are provided for the convenience of the reader and are not intended to be limiting with respect to the claimed subject matter.

Presented herein are methods and systems for using genotyping data of an individual to create and/or modify in-game avatars. In certain embodiments, genotyping data is used to create or generate an exportable avatar that at least partially reflects quantitative measurements in the genotyping data. Genotyping data is used to determine standardized values that characterize an individual's traits in a predefined standardized set of features that can be exported for import into a game to generate an avatar. Avatars may appear to physically resemble an individual or may have an appearance that fits the thematic elements of a game while still reflecting the phenotype of the individual (based on the individual's genotyping data). In certain embodiments, avatars that reflect an individual's biological traits are automatically generated from avatar objects that store data derived from genotyping data.

Genotyping data is generated from one or more biological samples provided by an individual. Genotyping data includes data from a plurality of measurements of an individual's genome. Genotyping data may include, in particular, data from measurements of particular single nucleotide polymorphisms (SNPs). The variant of a particular SNP that an individual has influences or determines a biological trait of an individual. Physical, behavioral, and health (e.g., disease) traits of an individual are determined or influenced by the SNP variants of the individual. In some embodiments, genotyping is performed using a PCR-based SNP genotyping assay to generate genotyping data.

In certain embodiments, genotyping data generated from biological samples is compiled into a personal genetic profile assessment. Personal genetic profile assessments may be presented to individuals using an assessment graphical user interface (“assessment GUI”). In certain embodiments, in order to provide an individual not only with their personal genetic profile assessment, but also convey information related to the particular traits and characteristics that are influenced by the specific SNP variants present in their genetic material in an organized and intuitive fashion, a framework comprising an intuitive hierarchical organization of data structures is used. The framework provides for storing relationships (e.g., associations) between particular SNPs, health-related traits and characteristics, and general classes of such health-related traits and characteristics, based on the specific characteristic that each particular SNP influences. Personal genetic profile assessments and the framework comprising an intuitive hierarchical organization of data structures are also described in PCT/US17/67264, filed Dec. 19, 2017, the contents of which is hereby incorporated by reference in their entirety.

In certain embodiments, the framework of a personal genetic profile assessment provides a convenient basis for creating or generating avatar objects for individuals. The data already stored in a personal genetic profile assessment (e.g., for use in providing an individual with her/his genomic information) can be associated (e.g., easily or quickly) with an avatar object or any sub-structures thereof. In certain embodiments, a personal genetic profile assessment or similarly organized hierarchy of data structures is used to store genotyping data for generating avatars without being additionally being made available to an individual to view (e.g., in an assessment GUI).

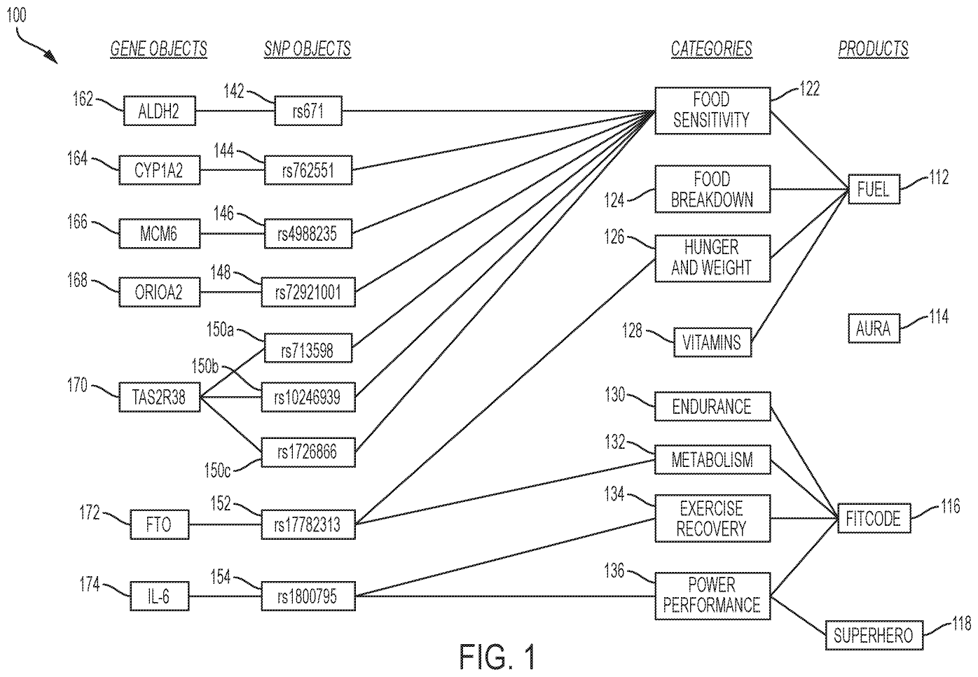

Turning to FIG. 1, in certain embodiments, a first class of data structures, referred to herein as products, are used to represent different general classes of traits and characteristics. In certain embodiments, a product data structure corresponds to a particular assessment ordered (e.g., purchased by the individual), in which unique versions of genes and/or SNPs that an individual has that influence the particular general class of health-related traits and characteristics that the corresponding product represents are identified (e.g., via genotyping measurements). In certain embodiments, each product has a name (e.g., a product data structure comprises a name (e.g., text data representing the name)) that provides a convenient, and memorable way to refer to the product. For example, a particular product 112 (e.g., named “FUEL™”) is used to represent a class of traits corresponding to the way in which an individual's body processes different foods and nutrients. Another product 114 (e.g., named “AURA™”) is used to represent a class of traits corresponding to skin health. Another product 116 (e.g., named “FITCODE™”) is used to represent a class of traits corresponding to physical fitness. Another product 118 (e.g., named “SUPERHERO™”) is used to represent a class of traits corresponding to physical and intellectual performance. In certain embodiments, such classes correspond to different aspects of an individual's avatar, such as the avatar's physical appearance or certain gameplay traits.

In certain embodiments, each product is in turn associated with one or more of a second class of data structures, referred to as categories. In certain embodiments, each category corresponds to a particular health-related trait or characteristic (e.g. food sensitivity, food breakdown, hunger and weight, vitamins, skin uv sensitivity, endurance, metabolism, joint health, muscle strength, intelligence). In certain embodiments, the categories with which a particular product is associated each correspond to different health-related traits or characteristics that are related to the general class of health-related traits or characteristics to which the particular product corresponds (e.g., the general class of health-related traits or characteristics that the product represents). As with products, in certain embodiments, each category has a name (e.g., a category data structure comprises a name (e.g., text data representing the name)) that provides a convenient, and memorable way to refer to the category.

In turn, each category is associated with one or more SNP objects, each SNP object corresponding to a specific SNP. Each SNP object associated with a particular category corresponds to a specific SNP that influences a specific health-related phenotype that relates to the trait or characteristic to which the particular category corresponds. Each SNP object may identify the specific SNP to which it corresponds via a SNP reference that the SNP object comprises. The SNP reference may be an alphanumeric code such as an accepted name of the SNP or other identifying mark or label capable of being stored electronically. The SNP reference may be an alphanumeric code such as a National Center for Biotechnology Information (NCBI) database reference number.

For example, the schematic of FIG. 1 shows an example of series of products, categories, and SNP objects that are associated with each other. Associated gene objects, to be described in the following, are also shown. The different products and categories are identified by their particular names, and the SNP objects each are identified by a respective SNP reference each comprises. In the example of FIG. 1, the SNP references are NCBI database reference numbers.

The “FUEL™” product 112 is associated with categories such as “Food Sensitivity” 122, “Food Breakdown” 124, “Hunger and Weight” 126, and “Vitamins” 128. Several SNP objects corresponding to specific SNPs that influence phenotypes related to an individual's sensitivity to different types of foods, and, accordingly, are associated with the “Food Sensitivity” category 122 are shown. In FIG. 1, the lines connecting the SNP objects to different categories indicate the association of each particular SNP object with one or more different categories.

For example, SNP object 142 corresponds to the rs671 SNP, which influences the manner in which an individual processes alcohol. In particular, depending on the particular variant of the rs671 SNP that an individual has, the individual may process alcohol normally, or be impaired in their ability to process alcohol, and likely suffer from adverse effects resulting from alcohol consumption, such as flushing, headaches, fatigue, and sickness. Accordingly, providing an individual with knowledge of the particular variant of the rs671 SNP that they have may allow them to modify their behavior accordingly, for example, by being mindful of the amounts of alcohol that they consume (e.g., on a regular basis, e.g., in social settings).

Other SNP objects corresponding to SNPs that influence food sensitivity related phenotypes, and, accordingly, are associated with the “Food Sensitivity” category 122 are shown. For example, SNP object 144 corresponds to the rs762551 SNP that influences caffeine metabolism, SNP object 146 corresponds to the rs4988235 SNP that influences lactose intolerance, and SNP object 148 corresponds to the rs72921001 SNP that influences an aversion to the herb Cilantro (e.g., depending on the particular variant of this SNP that an individual has, they may either perceive Cilantro as pleasant tasting, or bitter and soap-like in taste).

In certain examples, multiple SNPs are associated with a particular phenotype and, accordingly, the SNP objects to which they correspond may be grouped together. For example, three SNPS—rs713598 (corresponding to SNP object 150a), rs10246939 (corresponding to SNP object 150b), and rs1726866 (corresponding to SNP object 150c), —influence the sensitivity of an individual to bitter tasting foods (e.g., cabbage, broccoli, cauliflower, kale, brussel sprouts, and collard greens), and, accordingly, their enjoyment of or aversion to such foods.

SNPs correspond to specific locations within or nearby (e.g., a SNP may occur in a promotor region that influences transcription of a particular gene (e.g., a SNP may occur within 5 kb upstream or downstream of a particular gene, e.g., a SNP may occur within 100 kb upstream or downstream of a particular gene, e.g., a SNP may occur within 500 kb upstream or downstream of a particular gene, e.g., a SNP may occur within 1 Mb upstream or downstream of a particular gene) genes in an individual's genetic material. Accordingly, in certain embodiments, as shown in FIG. 1, each SNP object is associated with a gene object that corresponds to the particular gene within or nearby to which the SNP to which the SNP object corresponds is present. For example, the rs671 SNP corresponds to a location within the ALDH2 gene; the rs762551 SNP corresponds to a location within the CYP1A2 gene, the rs4988235 SNP occurs within the MCM6 gene, and the rs72921001 SNP occurs within the OR10A2 gene. Accordingly, SNP object 142 (corresponding to the rs671 SNP) is associated with gene object 162 (corresponding to the ALDH2 gene). Similarly, SNP object 144 (corresponding to the rs762551 SNP) is associated with gene object 162 (corresponding to the CYP1A2 gene), SNP object 146 (corresponding to the rs4988235 SNP) is associated with gene object 166 (corresponding to the MCM6 gene) and SNP object 148 (corresponding to the rs72921001 SNP) is associated with gene object 168 (corresponding to the OR10A2 gene).

Other SNP objects correspond to SNPs that are nearby particular genes of interest and thereby influence phenotypes associated with expression of the gene. For example, rs12696304 is a SNP that lies 1.5 kb downstream from the TERC gene, and influences biological aging associated with the TERC gene. Accordingly, in one example, a SNP object corresponding to the rs12696304 SNP is associated a gene object corresponding to the TERC gene.

In certain embodiments, multiple SNPs of interest occur within a single gene. For example, the three SNPs related to bitter taste—rs713598, rs10246939, and rs1726866—occur within the TAS2R38 gene. Accordingly, SNP objects 150a, 150b, and 150c, which correspond to the rs713598, rs10246939, and rs1726866 SNPs, respectively, are all associated with a gene object 170 corresponding to the TAS2R38 gene.

In certain embodiments, different products correspond to different general classes of health-related traits and characteristics. For example, products may be based on particular organs (e.g., product 114, named “AURA™”, is related to skin health), or particular habits, activities, or bodily functions. For example, food related biological characteristics and traits may be covered by a single products or a plurality of products. A single product or a plurality of products may be based on learning and brain function characteristics. For example, a product “BLISS” relates to cognitive and behavioral characteristics such as addiction, feelings, behavior (e.g., impulsiveness), and tolerance. A single product or a plurality of products may be based on physical fitness (e.g., cardiovascular strength, agility, flexibility, muscular strength).

For example, as shown in FIG. 1, another product 116 (e.g., named “FITCODE™”), relates to a general class of physical fitness related traits, and, accordingly, comprises categories associated with endurance 130 (“Endurance”), metabolism 132 (“Metabolism”), the ability of an individual to recover effectively following exercises 134 (“Exercise Recovery”), and cardiovascular fitness and skeletal muscle makeup 136 (“Power Performance”).

In certain embodiments, a particular SNP object is associated with two or more categories. For example, the rs17782313 SNP, occurring in the FTO gene, influences an individual's appetite. Accordingly, as shown in FIG. 1, the SNP object 152 corresponding to the rs17782313 SNP is associated with both the “Hunger and Weight” category 126 of the “FUEL™” product, and the “Metabolism” category 132 of the “FITCODE™” product. SNP object 152 is also associated with gene object 172, reflecting the fact that the rs17782313 SNP occurs in the FTO gene. In certain embodiments, as with the rs17782313 SNP object, each of a first category and a second category with which a particular SNP object is associated are associated with a different product. In certain embodiments, a particular SNP object is associated with a first category and a second category, and both the first category and the second category are associated with the same product.

For example, the SNP object 154 corresponding to the rs1800795 SNP of the IL-6 gene (accordingly, SNP object 154 is associated with gene object 174, which corresponds to the IL-6 gene) is associated with the “Exercise Recovery” category 134 and the “Power Performance” category 136, both of which are associated with the “FITCODE™” product 116. In addition, in certain embodiments, a category is associated with two or more products. For example, the “Power Performance” category 136 is associated with the “FITCODE™” product 116, as well as the “SUPERHERO” product 118, which provides an assessment of a general class of traits related to physical and intellectual performance.

Thus, by providing a framework comprising a hierarchical organization of data structures corresponding to products, categories, SNP objects, and gene objects, the frameworks described herein provide an intuitive and flexible approach to storing, updating, and creating new associations between different classes of health-related traits and characteristics, and the underlying genetic variations corresponding to different specific SNPs that influence them in order to store genotyping data for use in creating avatars.

In certain embodiments the hierarchical organization of product, category, SNP object and gene object data structures serves as a flexible template that facilitates both the rapid creation of individual personal genetic profile assessments from genotyping measurements taken from a plurality of individuals, and the use of personal genetic profile assessments in creating avatars for games. In particular, an individual may purchase or be given access to partial assessments corresponding to different products, in order to add, improve, or unlock certain features of his/her avatar in a game. Accordingly, an individual's personal genetic profile assessment corresponding to one or more products comprises, for each specific SNP associated with each category that is associated with each of the one or more products, an identification of the particular variant of the specific SNP that the individual has. Typically, the identification is obtained via one or more genotyping measurements performed on a biological sample taken from the individual (e.g., a blood sample, e.g., a cheek swab sample, e.g., a saliva sample).

In certain embodiments, an individual may purchase a first assessment corresponding to a first product, and provide a biological sample for genotyping. The individual's biological sample may be stored (e.g., cryogenically frozen). After a period of time, the individual may choose to purchase additional assessments corresponding to other products, and the individual's previously stored biological sample may be taken from storage for additional genotyping measurements of the additional SNPs that are associated with the new products. Moreover, in certain embodiments, additional new products may be created over time, and new assessments corresponding to new products offered to and purchased by individuals. In certain embodiments, as new information related to the influence of new and/or existing SNPs on different specific health-related phenotypes is elucidated, new SNP objects and gene objects may be created, and new associations between them and new or existing categories and/or products established. In certain embodiments, existing personal genetic profile assessments of individuals are automatically updated to reflect new information. Thus, an individual's avatar, according to the systems and methods described herein, may change or come to include additional features over time.

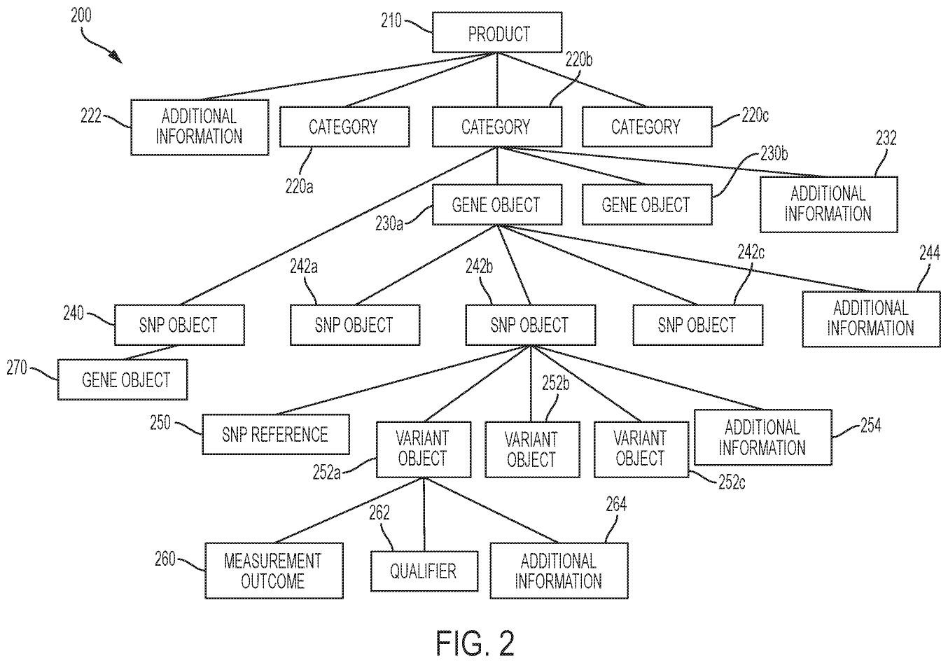

In certain embodiments, in order to facilitate the creation and presentation of individual personal genetic profile assessments (e.g., corresponding to one or more different products) based on the framework described above, the product, category, SNP object, and gene object data structures described herein store a variety of information. FIG. 2 is a block diagram of a hierarchy of data structures 200 of an example genetic profile product. An exemplary data structure of each type is shown to be associated with sub-data structures in order to simplify presentation of the figure. It is understood that data structures may be associated to any number of other data structures in the hierarchy if the association is consistent with the associations shown in FIG. 2. For example, category 220b is associated with gene objects 230a-b while category 220c may be associated with one or more gene objects and/or SNP objects, but any such associations are not shown. In some embodiments, data structures may be created without also forming associations between other structures of relevant types. For example, unassociated or partially associated data structures may be created for planning purposes such as during product or category development (e.g., category 220a has no associations yet because its scope has not been determined yet by the user). For example, unassociated or partially associated data structures may be created to allow genotyping data to be associated with relevant gene objects or SNP objects in order to retain the data in a ready to use format in the event that the gene objects and/or SNP objects are later associated with one or more categories.

Referring now to FIG. 2, product 210 comprises three categories 220a-c and additional information 222. Additional information 222 may be a name of the product, an icon associated with the product, and/or a description of the product. Category 220b comprises two gene objects 230a-b, one SNP object 240, and additional information 232. Additional information 232 may comprise a name of the category, a background image associated with the category, an icon associated with the category, a category order identifier, and/or a description of the category. SNP object 240 is associated with gene object 270. Gene object 230a is associated to three SNP objects 242a-c. Categories may be associated directly to SNP objects, such as category 220b is associated with SNP object 240, or they may be associated indirectly (e.g., SNP objects 242a-c are associated to category 220b via gene object 230a). The ability to form associations indirectly allows all SNP objects associated with a particular gene object to be associated with a category by forming a single association in cases where all SNP objects of a particular gene are relevant to a particular category. The ability to form associations directly allows a particular SNP object to be associated with a category without also forming an association with all other SNP objects associated with the gene object associated with the particular SNP object in cases where only one or a subset of SNP objects of a particular gene object are relevant to a category.

Gene object 230a is also associated with additional information 244. Additional information 244 may comprise one or more data structures comprising information such as a unique gene identifier that corresponds gene object 230a to a specific physical gene and descriptive information about the corresponding gene. The gene identifier may be an alphanumeric code such as an accepted name of the gene or other identifying mark or label capable of being stored electronically. Additional information may be stored as a single data structure or a plurality of data structures.

SNP object 242b is associated with SNP reference 250, and additional information 254. SNP reference 250 is a unique identifier of the SNP that corresponds the SNP object to a specific physical SNP. The SNP reference may be an alphanumeric code such as an accepted name of the gene or other identifying mark or label capable of being stored electronically. The SNP reference may be an alphanumeric code such as a National Center for Biotechnology Information (NCBI) database reference number. Additional information 254 may comprise one or more data structures with other descriptive information about the corresponding SNP.

Variants of a particular SNP can be represented within a corresponding SNP object using various combinations of data elements such as a measurement outcomes, and qualifiers. For example, a particular variant of a SNP can be identified by a measurement outcome, which is an identifier, such as an alphanumeric code, that identifies the specific alleles corresponding to the particular variant. For example, a measurement outcome such as the string “CC” identifies a first variant of the rs762551 SNP in which an individual has a cytosine (C) at the rs762551 position in each copy of their genetic material. A measurement outcome such as the string “AC” identifies a second variant of the rs762551 SNP in which an individual has a C in one copy and an adenine (A) in the other at the rs762551 position. A measurement outcome such as the string “AA” identifies a second variant of the rs762551 SNP in which an individual has an A at the rs762551 position in each copy of their genetic material. A qualifier is an identifier, such as an alphanumeric code, that identifies a classification of a variant, wherein the classification may be based on the prevalence of the variant within a population, a health-related phenotype associated with the variant, and/or other relevant classification bases. Additional information may also be included within a SNP object to describe a particular variant.

In certain embodiments, measurement outcomes and qualifiers that identify and classify, respectively the same variant are associated with each other to form a variant object associated with the SNP object. For example, variant object 252a comprises measurement outcome 260, qualifier 262. Variant object 252a is also comprises additional information 264. Additional information 264 comprises a description of the variant. For example, the additional information comprises a description of the specific health-related phenotype that an individual with the variant represented by variant object 252a exhibits or an explanation of the prevalence of the variant. A SNP object may be associated with a variant object to represent each variant of the particular SNP to which it corresponds. For example, SNP object is associated with three variant objects 252a-c.

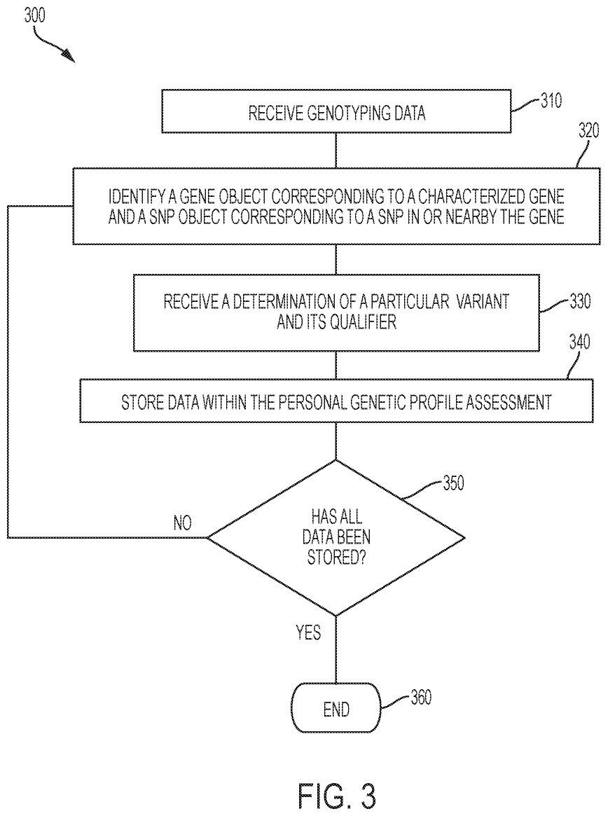

In order to populate an assessment GUI to provide it to an individual, genotyping data must be added to the individual's personal genetic profile assessment. FIG. 3 is a block diagram of a method 300 for adding genotyping data to an individual's personal genetic profile assessment. In step 310, a processor of a computing device receives genotyping data. In step 320, the processor identifies a gene object corresponding to a gene measured in the genotyping data and a SNP object corresponding to a SNP associated with the gene (e.g., the SNP occurring within the gene or occurring nearby the gene (e.g., within a promotor region that influences transcription of the gene, e.g., within 5 kb upstream or downstream of the gene, e.g., within 100 kb upstream or downstream of the gene, e.g., within 500 kb upstream or downstream of the gene, e.g., within 1 Mb upstream or downstream of the gene). In certain embodiments, genotyping data is stored as a table of data in a text file where each row corresponds to a unique SNP. In step 330, a particular variant of the SNP represented by the identified SNP object and its associated qualifier are determined based on data from genotyping measurements. For example, data corresponding to the measurement outcome of a particular variant may be stored as one or more columns at the end of each row. In step 340, the data is stored in the individual's personal genetic profile assessment. In step 350, the processor determines if all data of the genotyping data has been stored. If all data has not been stored in the individual's personal genetic profile assessment, then the method returns to step 320. If all data has been stored, then the method ends 360. In some embodiments, the processor determines if unstored data exists by determining if there is a row of data in the genotyping data below the just processed row.

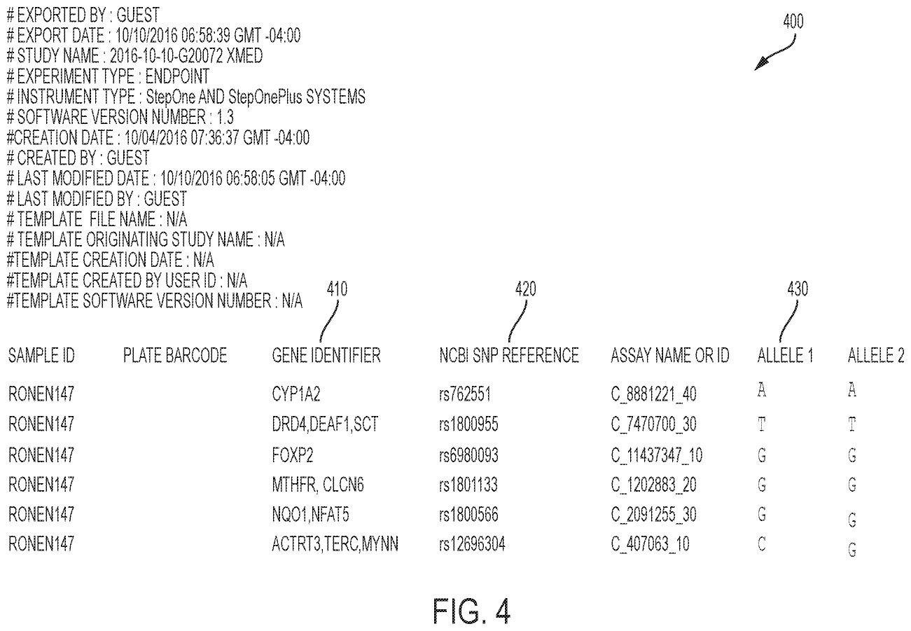

FIG. 4 shows exemplary genotyping data 400 that may be added to an individual's personal genetic profile assessment in accordance with method 300. Genotyping data may take the form of a text file saved by a user, wherein the text file is generated manually or as an output from equipment for performing genotyping measurements (e.g., TaqMan™ SNP genotyping assays). FIG. 4 comprises 6 rows of genotyping data from a single biological sample (“RONEN147”). Each row corresponds to data for a different SNP. Each SNP of genotyping data 400 is identified by at least a gene identifier 410 and a SNP reference 420. The gene identifier identifies the gene with which the SNP is associated. In certain embodiments, multiple (e.g., two or more) genes are associated with the SNP (e.g., the SNP may occur nearby two or more genes and influence phenotypes associated with each of the associated genes), and, accordingly, two or more corresponding gene identifiers are listed. Each SNP in the genotyping data has a corresponding variant identified by the allele measurements 430. The measurements “allele 1” and “allele 2” for a given SNP may be compared with measurement outcomes associated with the variants of a SNP object corresponding to the given SNP to populate an individual's personal genetic profile assessment.

The genotyping data in FIG. 4 used to populate an individual's personal genetic profile assessment is generated from one or more biological samples of the individual. However, the one or more biological samples used in populating an individual's personal genetic profile assessment may also be taken from a different human or a non-human animal. In some embodiments, genotyping data is generated from one or more biological samples of a non-human animal. For example, an individual may supply biological samples of a pet in order to use an avatar that reflects the phenotype of the pet (e.g., in a pet-care simulation or a monitoring program). In some embodiments, genotyping data is generated from one or more biological samples of a ward to whom the individual is a guardian. For example, parents may supply one or more biological samples to genotyping data for their child in order for the child to have an avatar that reflects the phenotype of the child.

Avatars are generated from avatar objects, data structures that define the features of an individual's avatar. Avatars generated from avatar objects are used in one or more of a plurality of digital environments relevant to an individual. For example, avatars may be used in a simulation, a virtual reality experience, a game (e.g., a video game), online matchmaking, interactive or realistic health tracking, simulated work environments, or similar digital environments. In certain embodiments, avatars for use in certain digital environments are graphically rendered based on data stored in avatar objects for individuals. In certain embodiments, avatars for use in certain digital environments are suitable for modeling or simulations (e.g., behavior modeling) based on data stored in avatar objects for individuals that characterizes their intangible traits (e.g., behavioral, cognitive, intellectual, or emotional traits).

As an example, an individual's avatar may be used in online matchmaking or group cohesion simulations. An avatar that reflects biological traits related to an individual's personality can be used in a simulation alongside avatars of one or more other individuals to determine pair or group dynamics (e.g., compatibility) based on the individuals' genotyping data. Such simulations (e.g., matching) can allow predictions (e.g., probabilistic predictions) to be made for certain scenarios such as romantic matchmaking, workplace environment simulating, or psychological evaluations.

As a further example, avatars of individuals may be generated based on genotyping data for the individuals for use in a virtual league. Genotyping data of individuals is used to create a plurality of avatar objects based on the physical characteristics and traits of the individuals determined from the genotyping data. The avatars for the individuals can be used to run simulated sports games in a virtual league. In this way, avatars for amateur or professional sportspeople are generated based on their genotyping data such that a group of friends, analysts, or competitors can play or simulate sports in a virtual league using the avatars. In certain embodiments, avatars for virtual leagues are generated from genotyping data that only relates to physical characteristics and traits and relevant cognitive characteristics and traits (e.g., to protect individuals from third parties discovering phenotypes of individuals for characteristics and traits irrelevant to the virtual league). The use of avatars based on genotyping data enhances the realism of the virtual league.

Avatar objects comprise substructures that correspond to different features of an individual's avatar. Features may be physical features, behavioral or ability (e.g., cognitive or athletic) features, health attributes, or a combination thereof. As examples, features that may be included in defining an individual's avatar, in certain embodiments, include hair color, sociability, muscular strength, and susceptibility to certain diseases. Features of an individual's avatar may be defined by data stored as numeric values, text strings, and/or alphanumeric strings. In certain embodiments, a framework of a hierarchy of data structures is used to define an individual's avatar object. Avatar objects may be exported or ported to a third party for use in generating or using the individual's avatar in a game created by the third party. In certain embodiments, the data stored in an avatar object obfuscates the identity (e.g., exact phenotype) of the individual to whom the avatar object corresponds (e.g., to alleviate privacy concerns related to third party use of genotyping data).

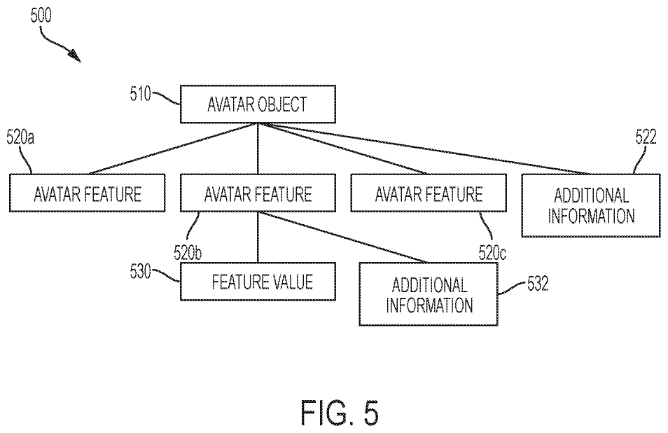

Referring now to FIG. 5, hierarchy 500 is an exemplary hierarchy of data structures used to generate an individual's avatar. Avatar object 510 comprises all the data created, generated, or stored based on the individual's genotyping data that is used to generate an individual's avatar. In certain embodiments, features of an individual's avatar are generated using generic or random options (e.g., feature values) when the individual's avatar object does not contain data corresponding to those particular features. For example, an individual's avatar object may not comprise data that determines hair color, so when generating the individual's avatar, a random hair color or generic hair color (e.g., black) is assigned or used. Avatar object 510 comprises avatar features 520a-c and additional information 522. Additional information 522 may be metadata, identification data (e.g., corresponding to an individual name or handle), or a set of data sub-structures that are used in generating an individual's avatar, but not directly related to a particular feature of the avatar. Avatar features 520a-c correspond to distinct features of an individual's avatar. In certain embodiments, avatar features correspond to distinct traits of an individual's phenotype that may be determined from genotyping data. In certain embodiments, a set of related avatar features may be associated with a sub-structure to which the avatar object is associated (e.g., wherein the sub-structure resides in a level between the avatar object level and avatar feature level in hierarchy 500). For example, all physical appearance related avatar features may be associated with a first sub-structure and all intellectually related avatar features may be associated with a second sub-structure, with both the first and second sub-structures being associated with an avatar object.

Avatar feature 520b comprises feature value 530 and additional information 532. For simplicity of presentation, avatar feature 520b is the only avatar feature in hierarchy 500 shown to have an associated feature value and additional information. Any avatar feature may have a feature value and additional information associated to it, in accordance with hierarchy 500. Additionally, complex features of an avatar may be defined for individuals by an avatar feature comprising a plurality of associated feature values. Feature value 530 defines the particular expression of the feature corresponding to avatar feature 520b that an individual's avatar has. Feature values may be stored as numeric values, text strings, and/or alphanumeric strings. In certain embodiments, feature values are integers within a certain predetermined range. For example, a feature value may be an integer within a range of 1-100 or within a range of 1-20 or within a range of 1-10. In certain embodiments, the feature value is a string that is used to generate an avatar, such as “brown,” “tall,” or “thin”. A numerical value may be used to indicate a physical trait. For example, a value of 1 can correspond to “tall” and a value of 0 can correspond to “short” for avatar feature “height.” In certain embodiments, different avatar features associated with an avatar object have associate feature values of different data types. For example, an avatar feature for hair color may have feature value “brown” associated with it, while an avatar feature for muscular strength may have feature value “80” (on a scale of 100) associated with it.

In certain embodiments, additional information 532 comprises any additional relevant information needed to generate an avatar in a game from an individual's avatar object. For example, additional information 532 may comprise a maximum value for the scale used in the corresponding feature value, an icon or image that corresponds to the feature value, a conditional statement or expression to adjust the feature value based on the data in other avatar features, or other similar information.

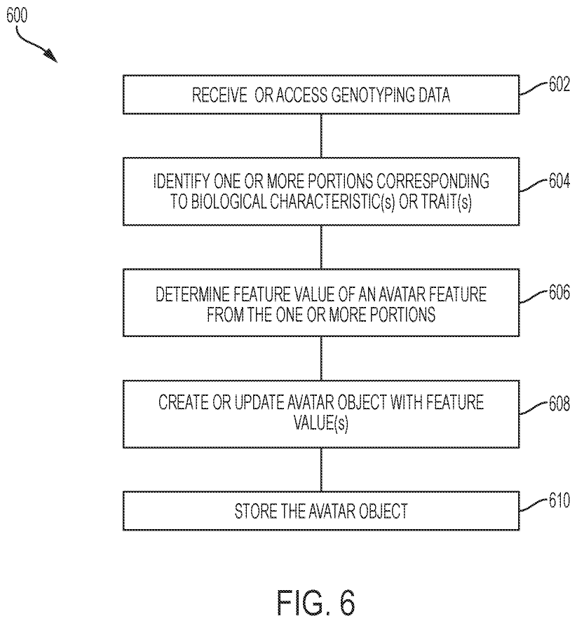

FIG. 6 shows an exemplary method for creating or modifying an individual's avatar object based on genotyping data. An avatar object created or modified according to method 600 may have a hierarchy according to or similar to hierarchy 500, or the avatar object may use another framework that allows values that define features of an individual's avatar to be stored within it. For example, in certain embodiments, avatar features and feature values are the same data structure. In step 602, genotyping data is received or accessed (e.g., by a processor of a computing device). In certain embodiments, genotyping data is received or accessed from a personal genetic profile assessment. In step 604, one or more portions of the genotyping data corresponding to biological characteristic(s) or trait(s) are identified. In certain embodiments, step 604 comprises identifying one or more SNP objects or variant objects in a personal genetic profile assessment and any genotyping data stored therein or associated thereto. In certain embodiments, raw (i.e., unprocessed or substantially unprocessed) genotyping data received in step 602 is searched directly to identify one or more portions in step 604.

In step 606, a feature value is determined for an avatar feature from the one or more portions of genotyping data identified in step 604. The determination is made using a determination method. The determination method comprise determining from a predefined rule or association. For example, variant objects corresponding to particular variants may be already associated with a particular feature value (e.g., by a developer that creates feature value rules or links feature values with biological traits) that is then assigned or associated to the corresponding avatar feature based on the one or more portions of genotyping data identified in step 604. As an additional illustrative example, a predefined rule may assign a random or predetermined feature value for an avatar feature based on a SNP object (or measurement of a SNP) received in the genotyping data, regardless of the particular variant of an individual. The determination method may be a probabilistic or pseudo-random method that is based on the one or more portions of genotyping data. For example, an individual with a particular variant of a SNP may be probabilistically or pseudo-randomly assigned a feature value for a particular avatar feature from the range of the possible feature value values. In some embodiments, a feature value is determined from a sub-range within the range that is defined based on the one or more portions of genotyping data.

In step 608, an avatar object is created or updated with the feature value and an association between the avatar object and the feature value (which may be a direct or indirect association). In certain embodiments, in step 608 or an equivalent step of a similar method, a new avatar feature data structure comprising the feature value is additionally created and associated in the avatar object. In certain embodiments, creating or updating an avatar object comprises associating a newly created feature value with a variant object in an individual's personal genetic profile assessment. In step 610, the created or updated (e.g., modified) avatar object is stored for further updating and/or retrieval (e.g., for use in game play). An avatar object may be updated based on a purchase of a new product or partial personal genetic profile assessment by an individual or an update to a previously purchased product or assessment made by a developer.

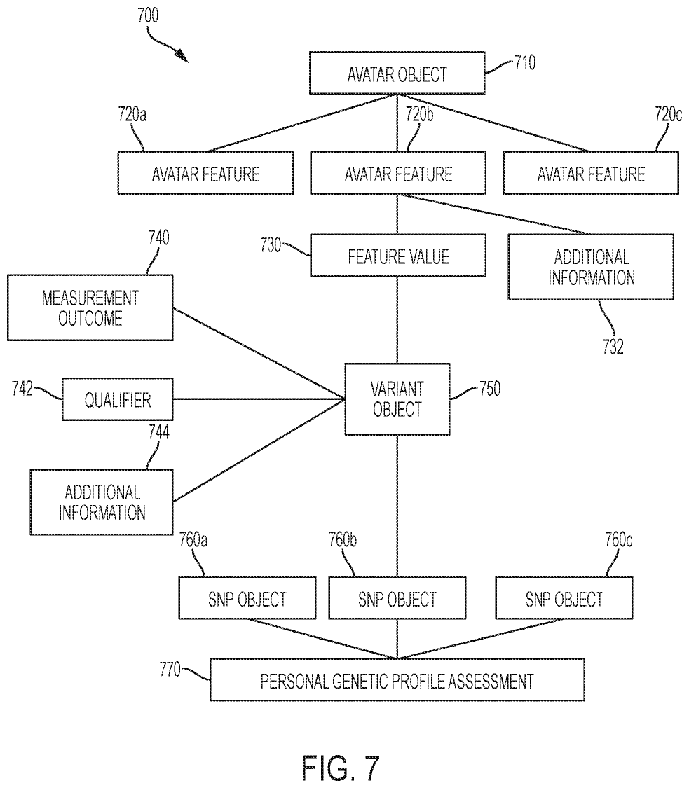

FIG. 7 illustrates an example of a relationship 700 between a hierarchy of data structures that define an individual's personal genetic profile assessment and a hierarchy of data structures that define an individual's avatar object that exists in certain embodiments. In FIG. 7, personal genetic profile assessment 770 and avatar object 710 both correspond to a single individual. For simplicity of presentation, of avatar features 720a-c, only avatar feature 720b is shown to have an associated feature value, feature value 730. Additionally, of SNP objects 760a-c, only SNP object 760b is shown to have an associated variant object, variant object 750. Avatar feature 720b is additionally shown to be associated with additional information 732. Variant object 750 is shown to be associated with measurement outcome 740, qualifier 742, and additional information 744. These associations and sub-structures exist in FIG. 7 for various reasons as discussed above.

In certain embodiments, a large generic framework of data structures in accordance with the associations illustrated in FIG. 7 is stored for each individual (e.g., player) who provides a biological sample. Frameworks may be generic in that they have defined data structures and associations, but null or default values. The various data structures in the framework may be filled in based on genotyping data when individuals purchase or are given access to them. For example, a generic avatar object may be created for each individual, but only personalized to each individual (e.g., by updating based on each individual's genotyping data) when each individual purchases their avatar (e.g., in one or more particular games). In certain embodiments, an individual may purchase particular avatar features or sets of avatar features individually. For example, an individual may only purchase access to portions of their avatar object that correspond to physical traits, such that they may have in-game avatars that resemble them, but do not have their behavioral or ability traits.

In certain embodiments, as a result of using a method to create or modify avatar objects, a series of associations between SNP object 760b, variant object 750, feature value 730, and avatar feature 720b exists. Prior to using the method, feature value 730 may be associated with variant object 750, wherein the value of feature value 730 is predefined based on variant object 750 or a sub-structure thereof (e.g., based on an individual's genotyping data). During the method, feature value 730 may be associated with avatar feature 720b. In this way, avatar features for traits characterized in an individual's personal genetic profile assessment are created or updated such that the individual's avatar generated using avatar object 710 has additional (or additionally defined) features.

Avatar features, feature values associated with avatar features, and relationships between feature values and genomic information (e.g., variants of a particular SNP) may be established by a developer using an avatar feature creation back-end (e.g., a creation graphical user interface (“creation GUI”)). In certain embodiments, a developer inputs data into a creation GUI using a plurality of graphical control elements, wherein the input data defines a generic avatar feature (e.g., one that is not yet reflective of a particular user's phenotype). The generic avatar feature may be personalized to an individual based on genotyping data for the individual. For example, one or more values of the avatar feature may be assigned based on portions of an individual's genotyping data or associated with other data structures created based on an individual's genotyping data.

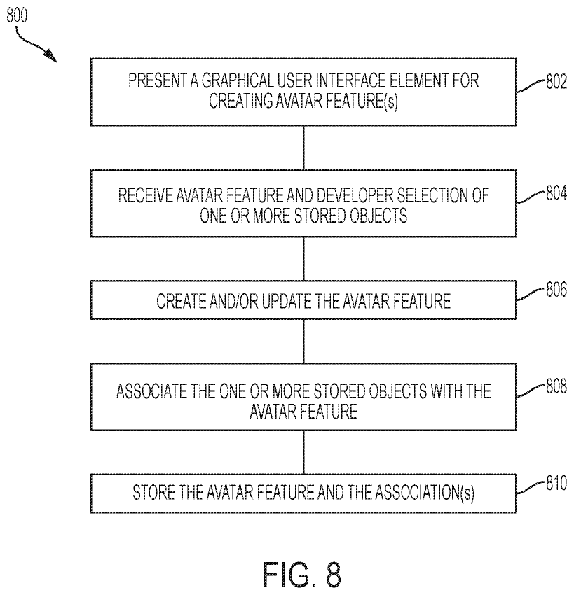

FIG. 8 shows an exemplary method 800 that may be performed in conjunction with a developer's use of a creation GUI to create avatar features. Exemplary method 800 starts at step 802. In step 802, a developer is presented with a graphical user interface element (e.g., a widget) (e.g., a creation GUI) for creating one or more avatar features. In certain embodiments, a graphical control element is provided in a creation GUI to allow a developer to simultaneously create additional avatar features. The developer provides input via one or more graphical control elements of the creation GUI that is used to create and/or update one or more avatar features. Additionally, in step 802, the creation GUI presented to the developer provides one or more graphical control elements that the developer can use to select one or more stored objects (e.g., variant objects, SNP objects, gene objects) that relate to avatar feature being created and/or updated. For example, the developer may select a SNP object that corresponds to a biological characteristic for which an avatar feature is being created.

In certain embodiments, a plurality of graphical control elements are provided to a developer for the developer to input a plurality of feature values of the avatar feature. The developer may select, for example, a plurality of variant objects from a list such that the variant objects correspond to the feature values as indicated by the developer in a creation GUI. In certain embodiments, a creation GUI allows a developer to input a mapping, function, or rule for determining one or more feature values for the avatar feature from each of the one or more stored objects. For example, each variant object selected by a developer could have an associated function that determines a feature value for the avatar feature based on the variant object.

In step 804, data input by the developer defining an avatar feature and selecting one or more stored objects, using the creation GUI presented in step 802, is received. In step 806, the received data is used to create and/or update an avatar feature. In certain embodiments, a developer can use a creation GUI to modify the data of a previously created avatar feature. In step 808, the one or more stored objects selected by the developer, and received in step 804, are associated with the avatar feature created and/or updated in step 806. For example, each variant object, of a set of selected variant objects, may be associated with a feature value input by the developer based on a correspondence indicated in a creation GUI by the developer. The one or more stored objects may be generic objects that are part of a personal genetic profile product or a generic personal genetic profile assessment (e.g., such that the association is used at a future time to personalize an avatar object based on a particular individual's personal genetic profile assessment). In step 810, the avatar feature created in step 806 and the association(s) generated in step 808 are stored for further updating and/or retrieval (e.g., to create a particular individual's avatar object).

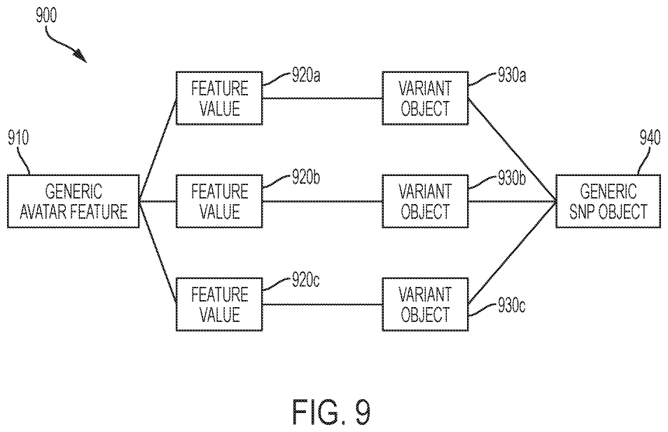

In certain embodiments, when creating generic avatar features, a developer provides a plurality of feature values that are then associated with a generic avatar feature. Each feature value may be associated with a particular variant object of a SNP object. As illustrated in FIG. 9, framework 900 shows the association between generic avatar feature 910 and generic SNP object 940. Generic SNP object 940 has three associated variant objects 930a-c that correspond to the three possible variants of the SNP corresponding to SNP object 940. Each variant object is associated with a feature value (e.g., variant object 930a with feature value 920a) of the three feature values 920a-c. Each feature value is associated with generic avatar feature 910. In certain embodiments, feature values 920a-c each comprise a function, mapping, or rule that determines the feature value. In this way, feature values can be probabilistic or pseudo-random. In certain embodiments, such an approach is easily scalable to a large number of individuals.

A generic framework comprising elements similar to, or in accordance with, framework 900 may be created based on input from a developer when creating avatar features. In this way, a developer can create one framework that automatically populates an individual's avatar object when an individual's genotyping data is associated or added to a generic personal genetic profile assessment. When an individual's personal genetic profile assessment is created or updated, the assessment will comprise only one of variant objects 930a-c because an individual can only have one variant for a particular SNP. Thus, the feature value associated with that variant object and the variant object will remain in the personalized framework and the remaining feature values for the avatar feature and the remaining variant objects for the SNP object will be deleted. All feature values not associated with the variant object corresponding to the variant of a trait that an individual has may be deleted when creating or updating an individual's avatar object.

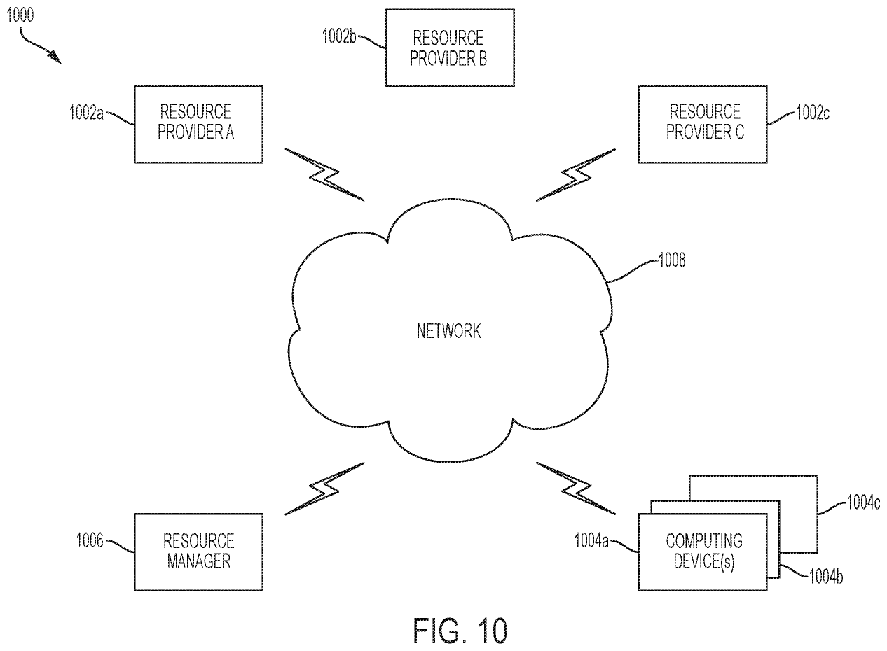

FIG. 10 shows an illustrative network environment 1000 for use in the methods and systems described herein. In brief overview, referring now to FIG. 10, a block diagram of an exemplary cloud computing environment 1000 is shown and described. The cloud computing environment 1000 may include one or more resource providers 1002a, 1002b, 1002c (collectively, 1002). Each resource provider 1002 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 1002 may be connected to any other resource provider 1002 in the cloud computing environment 1000. In some implementations, the resource providers 1002 may be connected over a computer network 1008. Each resource provider 1002 may be connected to one or more computing device 1004a, 1004b, 1004c (collectively, 1004), over the computer network 1008.

The cloud computing environment 1000 may include a resource manager 1006. The resource manager 1006 may be connected to the resource providers 1002 and the computing devices 1004 over the computer network 1008. In some implementations, the resource manager 1006 may facilitate the provision of computing resources by one or more resource providers 1002 to one or more computing devices 1004. The resource manager 1006 may receive a request for a computing resource from a particular computing device 1004. The resource manager 1006 may identify one or more resource providers 1002 capable of providing the computing resource requested by the computing device 1004. The resource manager 1006 may select a resource provider 1002 to provide the computing resource. The resource manager 1006 may facilitate a connection between the resource provider 1002 and a particular computing device 1004. In some implementations, the resource manager 1006 may establish a connection between a particular resource provider 1002 and a particular computing device 1004. In some implementations, the resource manager 1006 may redirect a particular computing device 1004 to a particular resource provider 1002 with the requested computing resource.

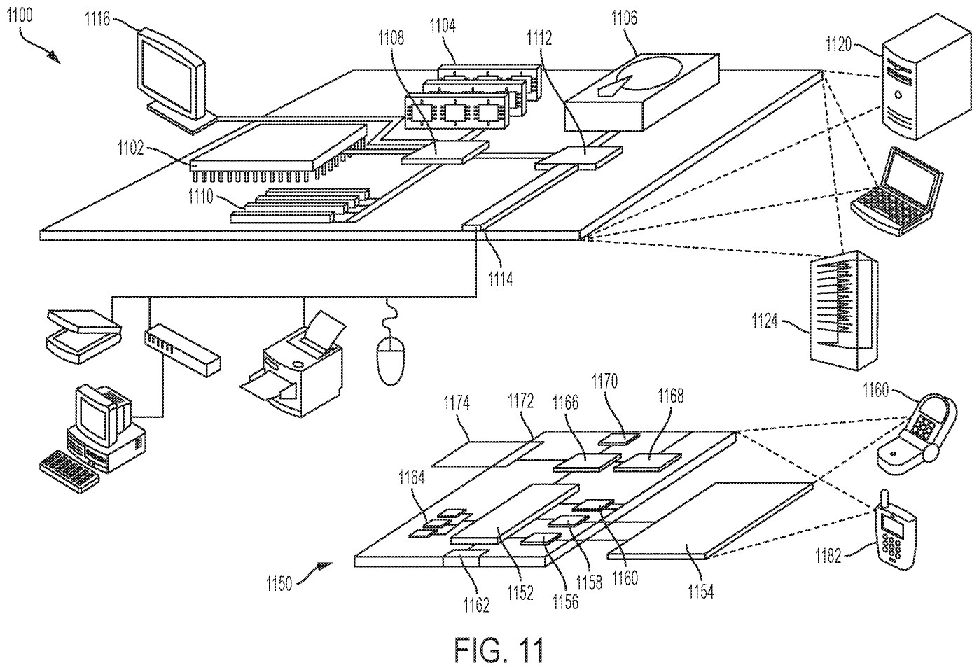

FIG. 11 shows an example of a computing device 1100 and a mobile computing device 1150 that can be used in the methods and systems described in this disclosure. The computing device 1100 is intended to represent various forms of digital computers, such as laptops, desktops, workstations, personal digital assistants, servers, blade servers, mainframes, and other appropriate computers. The mobile computing device 1150 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 1100 includes a processor 1102, a memory 1104, a storage device 1106, a high-speed interface 1108 connecting to the memory 1104 and multiple high-speed expansion ports 1110, and a low-speed interface 1112 connecting to a low-speed expansion port 1114 and the storage device 1106. Each of the processor 1102, the memory 1104, the storage device 1106, the high-speed interface 1108, the high-speed expansion ports 1110, and the low-speed interface 1112, are interconnected using various busses, and may be mounted on a common motherboard or in other manners as appropriate. The processor 1102 can process instructions for execution within the computing device 1100, including instructions stored in the memory 1104 or on the storage device 1106 to display graphical information for a GUI on an external input/output device, such as a display 1116 coupled to the high-speed interface 1108. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 1104 stores information within the computing device 1100. In some implementations, the memory 1104 is a volatile memory unit or units. In some implementations, the memory 1104 is a non-volatile memory unit or units. The memory 1104 may also be another form of computer-readable medium, such as a magnetic or optical disk.

The storage device 1106 is capable of providing mass storage for the computing device 1100. In some implementations, the storage device 1106 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 1102), perform one or more methods, such as those described above. The instructions can also be stored by one or more storage devices such as computer- or machine-readable mediums (for example, the memory 1104, the storage device 1106, or memory on the processor 1102).

The high-speed interface 1108 manages bandwidth-intensive operations for the computing device 1100, while the low-speed interface 1112 manages lower bandwidth-intensive operations. Such allocation of functions is an example only. In some implementations, the high-speed interface 1108 is coupled to the memory 1104, the display 1116 (e.g., through a graphics processor or accelerator), and to the high-speed expansion ports 1110, which may accept various expansion cards (not shown). In the implementation, the low-speed interface 1112 is coupled to the storage device 1106 and the low-speed expansion port 1114. The low-speed expansion port 1114, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 1100 may be implemented in a number of different forms, as shown in the figure. For example, it may be implemented as a standard server 1120, or multiple times in a group of such servers. In addition, it may be implemented in a personal computer such as a laptop computer 1122. It may also be implemented as part of a rack server system 1124. Alternatively, components from the computing device 1100 may be combined with other components in a mobile device (not shown), such as a mobile computing device 1150. Each of such devices may contain one or more of the computing device 1100 and the mobile computing device 1150, and an entire system may be made up of multiple computing devices communicating with each other.

The mobile computing device 1150 includes a processor 1152, a memory 1164, an input/output device such as a display 1154, a communication interface 1166, and a transceiver 1168, among other components. The mobile computing device 1150 may also be provided with a storage device, such as a micro-drive or other device, to provide additional storage. Each of the processor 1152, the memory 1164, the display 1154, the communication interface 1166, and the transceiver 1168, are interconnected using various buses, and several of the components may be mounted on a common motherboard or in other manners as appropriate.

The processor 1152 can execute instructions within the mobile computing device 1150, including instructions stored in the memory 1164. The processor 1152 may be implemented as a chipset of chips that include separate and multiple analog and digital processors. The processor 1152 may provide, for example, for coordination of the other components of the mobile computing device 1150, such as control of user interfaces, applications run by the mobile computing device 1150, and wireless communication by the mobile computing device 1150.

The processor 1152 may communicate with a user through a control interface 1158 and a display interface 1156 coupled to the display 1154. The display 1154 may be, for example, a TFT (Thin-Film-Transistor Liquid Crystal Display) display or an OLED (Organic Light Emitting Diode) display, or other appropriate display technology. The display interface 1156 may comprise appropriate circuitry for driving the display 1154 to present graphical and other information to a user. The control interface 1158 may receive commands from a user and convert them for submission to the processor 1152. In addition, an external interface 1162 may provide communication with the processor 1152, so as to enable near area communication of the mobile computing device 1150 with other devices. The external interface 1162 may provide, for example, for wired communication in some implementations, or for wireless communication in other implementations, and multiple interfaces may also be used.

The memory 1164 stores information within the mobile computing device 1150. The memory 1164 can be implemented as one or more of a computer-readable medium or media, a volatile memory unit or units, or a non-volatile memory unit or units. An expansion memory 1174 may also be provided and connected to the mobile computing device 1150 through an expansion interface 1172, which may include, for example, a SIMM (Single In Line Memory Module) card interface. The expansion memory 1174 may provide extra storage space for the mobile computing device 1150, or may also store applications or other information for the mobile computing device 1150. Specifically, the expansion memory 1174 may include instructions to carry out or supplement the processes described above, and may include secure information also. Thus, for example, the expansion memory 1174 may be provided as a security module for the mobile computing device 1150, and may be programmed with instructions that permit secure use of the mobile computing device 1150. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier and, when executed by one or more processing devices (for example, processor 1152), perform one or more methods, such as those described above. The instructions can also be stored by one or more storage devices, such as one or more computer- or machine-readable mediums (for example, the memory 1164, the expansion memory 1174, or memory on the processor 1152). In some implementations, the instructions can be received in a propagated signal, for example, over the transceiver 1168 or the external interface 1162.

The mobile computing device 1150 may communicate wirelessly through the communication interface 1166, which may include digital signal processing circuitry where necessary. The communication interface 1166 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 1168 using a radio-frequency. In addition, short-range communication may occur, such as using a Bluetooth®, Wi-Fi™, or other such transceiver (not shown). In addition, a GPS (Global Positioning System) receiver module 1170 may provide additional navigation- and location-related wireless data to the mobile computing device 1150, which may be used as appropriate by applications running on the mobile computing device 1150.

The mobile computing device 1150 may also communicate audibly using an audio codec 1160, which may receive spoken information from a user and convert it to usable digital information. The audio codec 1160 may likewise generate audible sound for a user, such as through a speaker, e.g., in a handset of the mobile computing device 1150. Such sound may include sound from voice telephone calls, may include recorded sound (e.g., voice messages, music files, etc.) and may also include sound generated by applications operating on the mobile computing device 1150.

The mobile computing device 1150 may be implemented in a number of different forms, as shown in the figure. For example, it may be implemented as a cellular telephone 1180. It may also be implemented as part of a smart-phone 1182, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

Certain embodiments of the present invention were described above. It is, however, expressly noted that the present invention is not limited to those embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

Having described certain implementations of methods and systems for generating and modifying avatars that reflect traits of individuals based on genotyping data, it will now become apparent to one of skill in the art that other implementations incorporating the concepts of the disclosure may be used. Therefore, the disclosure should not be limited to certain implementations, but rather should be limited only by the spirit and scope of the following claims.

What is claimed is:

1. A method for creating (and/or updating) an avatar object based on genotyping data, the method comprising:
receiving (and/or accessing), by a processor of a computing device, genotyping data generated using a biological sample of one or more individuals;
identifying, by the processor, one or more portions of the genotyping data that correspond to a distinct biological characteristic or trait;
determining, by the processor, an avatar feature and a feature value of the avatar feature based on the one or more portions of the genotyping data;
creating (and/or updating), by the processor, an avatar object for each of the one or more individuals, wherein the avatar object comprises each determined avatar feature and the feature value of the avatar feature for each of the one or more individuals; and
storing, by the processor, the avatar object for further use by a person.

2. The method of claim 1, comprising:
identifying, by the processor, at least one avatar feature that corresponds to at least one of the one or more portions of the genotyping data.

3. The method of any one of the preceding claims, wherein the genotyping data has been generated from one or more genotyping measurements of one or more SNPs.

4. The method of any one of the preceding claims, wherein the receiving (and/or accessing) step comprises accessing or extracting (e.g., by the processor) genotyping data from a personal genetic profile assessment.

5. The method of any one of the preceding claims, wherein the determining step comprises determining, by the processor, the feature value by selecting a numerical value from a predefined range of values.

6. The method of any one of the preceding claims, wherein the determining step comprises determining, by the processor, the feature value based on a plurality of the one or more portions of the genotyping data.

7. The method of any one of the preceding claims, comprising:
determining, by the processor, a sub-range from the one or portions of genotyping data, wherein the feature value is selected from the sub-range.

8. The method of any one of the preceding claims, wherein the determining step comprises automatically determining, by the processor, the feature value based on a particular variant of a SNP that the individual has.

9. The method of any one of the preceding claims, wherein the avatar object is for use in a member selected from the group consisting of playing a game, running a simulation, in online matchmaking, in digital health tracking, and a virtual reality experience.

10. The method of any one of the preceding claims, wherein the avatar object obfuscates identities of the one or more individuals.

11. A method for linking feature values of avatar features to personal genetic profile products, the method comprising:
presenting, by a processor of a computing device, a graphical user interface element for creation of an avatar feature that defines a portion of an avatar for use by an individual in a game;
receiving, by the processor, via the graphical user interface element, the avatar feature;
receiving, by the processor, via the graphical user interface element, a developer selection of one or more stored objects that correspond to one or more genomic constituents, wherein the one or more genomic constituents correspond to a biological trait corresponding to the avatar feature;
creating and/or updating, by the processor, the avatar feature;
associating, by the processor, the avatar feature with the one or more stored objects; and
storing, by the processor, the avatar feature and the association(s) between the avatar feature and the one or more stored objects for further retrieval and/or updating.

12. The method of claim 11, wherein the avatar feature comprises one or more feature values associated with the avatar feature, wherein the one or more feature values are used to graphically render an avatar from an avatar object comprising the avatar feature or to provide a quantitative value of an individual ability for use in game play.

13. The method of claim 12, comprising the step of:
associating, by the processor, each of the one or more feature values with a distinct variant object, wherein each distinct variant object is associated with a common SNP object.

14. The method of any one of claims 11-13, wherein the one or more stored objects are one or more SNP objects and the one or more genomic constituents are one or more SNPs.

15. The method of any one of claims 11-14, wherein the association(s) between the avatar feature and the one or more stored genomic objects are indirect association(s).

16. The method of any one of claims 11-15, comprising:
receiving, by the processor, a developer selection of one or more avatar feature collection objects, wherein the one or more avatar feature collection objects correspond to a set of related biological characteristics; and
associating, by the processor, the avatar feature with the one or more avatar feature collection objects.

17. A system for creating (and/or updating) an avatar object based on genotyping data, the system comprising:
a processor; and
a non-transitory computer readable medium having instructions stored thereon, wherein the instructions, when executed by the processor, cause the processor to:
receive (and/or access) genotyping data generated using a biological sample of one or more individuals;
identify one or more portions of the genotyping data that correspond to a distinct biological characteristic or trait;
determine an avatar feature and a feature value of the avatar feature based on the one or more portions of the genotyping data;
create (and/or update) an avatar object for each of the one or more individuals, wherein the avatar object comprises each determined avatar feature and the feature value of the avatar feature for each of the one or more individuals; and
store the avatar object for further use by a person.

18. The system of claim 17, wherein the instructions, when executed by the processor, cause the processor to identify at least one avatar feature that corresponds to at least one of the one or more portions of the genotyping data.

19. The system of any one of claim 17 or 18, wherein the genotyping data has been generated from one or more genotyping measurements of one or more SNPs.

20. The system of any one of claims 17-19, wherein the instructions, when executed by the processor, cause the processor to receive (and/or access) the genotyping data by accessing or extracting the genotyping data from a personal genetic profile assessment.

21. The system of any one of claims 17-20, wherein the instructions, when executed by the processor, cause the processor to determine the feature value by selecting a numerical value from a predefined range of values.

22. The system of any one of claims 17-21, wherein the instructions, when executed by the processor, cause the processor to determine the feature value based on a plurality of the one or more portions of the genotyping data.

23. The system of any one of claims 17-22, wherein the instructions, when executed by the processor, cause the processor to determine a sub-range from the one or portions of genotyping data, wherein the feature value is selected from the sub-range.

24. The system of any one of claims 17-23, wherein the instructions, when executed by the processor, cause the processor to determine the feature value based on a particular variant of a SNP that the individual has.

25. The system of any one of claims 17-24, wherein the avatar object is for use in a member selected from the group consisting of playing a game, running a simulation, in online matchmaking, in digital health tracking, and a virtual reality experience.

26. The system of any one of claims 17-25, wherein the avatar object obfuscates identities of the one or more individuals.

27. A system for linking avatar features to stored genomic objects, the system comprising:
a processor; and
a non-transitory computer readable medium having instructions stored thereon, wherein the instructions, when executed by the processor, cause the processor to:
present a graphical user interface element for creation of an avatar feature that defines a portion of an avatar for use by an individual in a game;
receive, via the graphical user interface element, the avatar feature;
receive, via the graphical user interface element, a developer selection of one or more stored objects that correspond to one or more genomic constituents, wherein the one or more genomic constituents correspond to a biological trait corresponding to the avatar feature;
create and/or update the avatar feature;
associate the avatar feature with the one or more stored objects; and
store the avatar feature and the association(s) between the avatar feature and the one or more stored objects for further retrieval and/or updating.

28. The system of claim 27, wherein the avatar feature comprises one or more feature values associated with the avatar feature, wherein the one or more feature values are used to graphically render an avatar from an avatar object comprising the avatar feature or to provide a quantitative value of an individual ability for use in game play.

29. The system of claim 28, wherein the instructions, when executed by the processor, cause the processor to associate each of the one or more feature values with a distinct variant object, wherein each distinct variant object is associated with a common SNP object.

30. The system of any one of claims 27-29, wherein the one or more stored objects are one or more SNP objects and the one or more genomic constituents are one or more SNPs.

31. The system of any one of claims 27-30, wherein the association(s) between the avatar feature and the one or more stored genomic objects are indirect association(s).

32. The system of any one of claims 27-31, wherein the instructions, when executed by the processor, cause the processor to:
receive a developer selection of one or more avatar feature collection objects, wherein the one or more avatar feature collection objects correspond to a set of related biological characteristics; and
associate the avatar feature with the one or more avatar feature collection objects.
